(19) United States
(12) Patent Application Publication
(10) Pub. No.: US 2011/0201100 A1
(43) Pub. Date: Aug. 18, 2011

(54) SINGLE USE CELL CULTURE BIOREACTOR MANIFOLD SYSTEM

(75) Inventors: Stephen P. Proulx, Boxboro, MA (US); James E. Kelly, JR., Melrose, MA (US); Adriane Harrington, Concord, MA (US); Joeseph William Muldoon, Berlin, MA (US)

(73) Assignee: Millipore Corporation, Billerica, MA (US)

(21) Appl. No.: 13/009,470

(22) Filed: Jan. 19, 2011

Related U.S. Application Data
(60) Provisional application No. 61/336,226, filed on Jan. 19, 2010.

(51) Int. Cl. C12M 1/34; C12M 1/00; C12M 1/02

(52) U.S. Cl. 435/288.7; 435/289.1; 435/287.1

(57) ABSTRACT

The invention provides a disposable cell culture bioreactor manifold system for use in coupling sensors, fluid samplers, conduits, and the like, to a cell culture bioreactor in a sterile manner. The disposable bioreactor manifold system includes an externally attachable bioreactor manifold connector body for fluidly attaching modular sensor arrangements that measure physical variables and other parameters of medium contained within a bioreactor, as well as medium sampling components, and other connections, as well as at least one conduit fluidly connecting connecting the bioreactor manifold connector body with a pump for pumping fluids between the bioreactor and the bioreactor manifold connector body.

### FIELD OF THE INVENTION

The present invention relates to methods and a system for processing biological materials, and more particularly, to disposable components and systems for processing biological materials.

### BACKGROUND OF THE INVENTION

A variety of vessels for manipulating biological materials and fluids, and for carrying out chemical, biochemical and/or biological reactions, as well as used for sterile and non-sterile mixing applications are available. Modern cell cultivation is typically accomplished using a bioreactor or a fermentor vessel. Despite the fact that a bioreactor and a fermentor are essentially similar in design and general function, the dichotomy in nomenclature is sometimes used to distinguish between animal and plant cell culture. Herein we will use the term bioreactor in an inclusive, generic sense as including both aerobic and anerobic cultivation of both microbial, animal, insect and plant cells, and thus encompassing a fermentor.

Traditional bioreactors are typically designed as stationary pressurized vessels. While disposable single use bioreactors (hereinafter referred to as “SUBs” or “SUB”) utilize plastic sterile bags. Which ever type of bioreactor design is selected, the bioreactor's chemical, biochemical, nutritional, biological and environmental conditions like gas (i.e., air, oxygen, nitrogen, carbon dioxide, ammonium,) flow rates, temperature, pH and dissolved oxygen levels, and agitation speed/circulation rate need to be closely monitored by sensors and controlled in order to provide ongoing optimum conditions within the bioreactor, such that microorganisms, cells, and the like are able to perform their desired functions successfully.

Disposable SUB technology can be configured from the manufacturer in multiple, flexible configurations, and offer advantages over the classic designs utilizing glass and stainless steel in the culture of cells, such as a lower capital investment, lower validation, lower risk of cross contamination and less supporting infrastructure.

However, current disposable SUBs do not include a total disposable system, because components such as probes, sensors and the like are generally reused, requiring sterilization prior to each repeated use. Thus, current state of the art disposable bioreactor systems are not efficient, especially when it comes to mixing, and have a lag time between uses so that probes, sensors and/or other components may be sterilized prior to another use.

In addition, disposable SUBs are usually sterilized by the manufacturer with gamma radiation or very aggressive chemicals such as ethylene oxide (ETO). A problem frequently arises in that many sensor systems, in particular the sensor electronics, do not withstand such a sterilization step.

Ports are a necessary feature of SUBs for delivering controlled volumes of gas, liquid, or other material to growth media containing cells; for sampling sample fluid out of the bioreactor; for extracting material out of the bioreactor; and for inserting probes, such as a temperature probe, to monitor conditions within the SUB.

Clearly, it is of the utmost importance to monitor dissolved oxygen, pH, carbon dioxide, glucose, ammonium, and specific protein levels, and any number of other parameters either required or produced by cellular metabolism inside the SUB.

Current designs are limited however, in the number and manner with which sensors or the like can be installed on a SUB in order to actively control the bioreactor. This is caused by the complexity of inserting pre-sterilized probes and sensors into the sterile environment of the SUB. In addition, it is impractical to attach a large number of sterile to sterile connections onto a SUB, and the financial cost for attaching a large number of sterile to sterile connections onto a SUB is prohibitively expensive.

Each connection into the bioreactor increases the likelihood of contamination. Typical current systems allow a maximum of four insertion points into the bioreactor. However, in GMP manufacturing environments it is often required to have a redundant sensor in case of failure or drift.

Conventional ports comprise tubular metal or hard plastic stems that are permanently attachable to the bioreactor container. Various tubes or probes are then attached to the ports or are passed through the ports. Great care must be taken so that no leaking or contamination occurs at the ports. Although conventional ports are useful for their intended purpose, they have a number of shortcomings.

For example, because conventional ports typically are made of metal or hard plastic, the ports are typically rigid and inflexible. Because of this inflexibility, it can be difficult to establish a seal around tubes or other structures that are passed through the ports. As a result, an unwanted dead space can be formed between the ports and the structures passing there through.

Furthermore, the inflexibility of conventional ports can cause problems when used with flexible disposable containers. Rigid ports decrease the flexibility of the containers and increase the risk that the ports could damage the containers.

Although SUB systems and other fluid manipulating, sampling, and sensor monitoring systems are known, improvements to such systems would be beneficial. Accordingly, what is needed is an improved manner in which scientists and researchers can actively monitor and control any number of parameters inside any number of different SUBs, without increasing the complexity of the SUBs, as well as decreasing the number of connections into the bioreactor so as to diminish the likelihood of contamination when pre-sterilized probes and sensors are inserted into the sterile environment of the bioreactor.

### SUMMARY OF THE INVENTION

The invention as taught herein provides a disposable single use cell culture bioreactor manifold, system and methods of using the same.

The disposable single use cell culture bioreactor manifold system provided herein is assembled by configuring any number of modular components, such as connections, sensors, samplers, additional lines, and conduits, fluidly connected or coupled to a bioreactor manifold connector body which can be externally attached and fluidly connected to a port on a bioreactor, either directly or indirectly by way of a conduit or the like, in order to interact with a liquid medium flowing through the manifold connector body. The system advantageously simplifies design, lessens the possibility of contamination of the bioreactor, and lowers the cost of monitoring, testing and supporting of bioreactor vessels.

In certain embodiments, the invention provides a disposable bioreactor manifold connector body coupled by way of a preexisting port to a bioreactor and a fluid circulation connector body and pumping system that fluidly connects the bioreactor and disposable bioreactor manifold connector body such that media flows from the bioreactor to the manifold connector body and back to the bioreactor. Modular components, are preferably disposable, and include connections, sensors, probes, samplers, additional lines and combinations thereof, coupled to the manifold connector body via a series of sterile manifold ports in order to interact with the medium flowing through the manifold connector body.

In other embodiments, the invention provides a disposable bioreactor manifold system having i) a modular sensor arrangement coupled to one or more manifold ports on the bioreactor manifold connector body for measuring and monitoring various parameters within the bioreactor and media, ii) a recirculating pump for pumping fluids from the bioreactor to the bioreactor manifold connector body, and vice versa, iii) at least one conduit fluidly connecting the bioreactor to the recirculating pump, iv) at least one conduit fluidly connecting the bioreactor manifold connector body to the recirculating pump, and v) at least one conduit fluidly connecting the bioreactor manifold connector body to the bioreactor.

In still another embodiment the invention provides a bioreactor manifold system having i) a modular fluid sampler coupled to the bioreactor manifold connector body for sampling the medium contained within the bioreactor as the medium flows through the manifold connector body, ii) a recirculating pump for pumping fluids from the bioreactor to the bioreactor manifold connector body and vice versa, iii) at least one conduit fluidly connecting the bioreactor to a recirculating pump, iv) at least one conduit fluidly connecting the bioreactor manifold connector body to the recirculating pump, and v) at least one conduit fluidly connecting the bioreactor manifold connector body to the bioreactor.

In yet another embodiment, the invention provides a bioreactor manifold system having a bioreactor manifold connector body, one or more pumps, one or more conduits, one or more additional modular connections, sensors, or samplers that are each pre-sterilized and/or preassembled.

Some embodiments of the invention provide a bioreactor manifold system having a bioreactor manifold connector body, one or more pumps, one or more conduits, one or more additional modular connections, sensors, or samplers that are each provided as individual modular components which must be assembled and/or subsequently autoclaved/sterilized by the user.

In yet another embodiment, the invention provides a method of using a bioreactor manifold system by recirculating a medium via a pumping connector body, such as a vane pump, diaphragm pump or peristaltic pump, between a bioreactor and a bioreactor manifold connector body having one or more modular connections, sensors, or samplers coupled to one or more ports on the manifold connector body, in order to interact with the medium flowing through the manifold connector body.

In still another embodiment the invention provides a bioreactor manifold system having a single use disposable pump that maintains the volume and flow rate inside a bioreactor manifold connector body which accurately represents and maintains the volume and flow rate of the bioreactor, such that the bioreactor manifold connector body does not result in dead zones where particulates can build up, and the process of pumping the medium and cells does not adversely effect production of a protein, biological sample, or other culture of interest.

In certain embodiments, the present invention provides a bioreactor manifold system capable of recirculating medium within a bioreactor manifold connector body by air pressure, a mixing impeller, or any other means of creating flow, so long as the volume and flow rate inside the bioreactor manifold connector body accurately represents and maintains the volume and flow rate of the bioreactor, such that the bioreactor manifold does not result in dead zones where particulate or the like can build up, and the process of pumping the cells and medium through the manifold connector body does not adversely effect production of a protein, biological sample or other culture of interest.

Some embodiments of the invention provide a bioreactor manifold system having a partial recirculation component in order to reduce the number of connections between the bioreactor and the bioreactor manifold connector body.

In yet further embodiments the invention provides a bioreactor manifold system having hollow fibers or other porous materials or filters incorporated in the manifold connector body to facilitate the exchange of culture media, or harvest proteins, biological samples, or cultures of interest.

Additional features and advantages of the invention will be set forth in the detailed description which follows. Many modifications and variations of this invention can be made without departing from its spirit and scope, as will be apparent to those skilled in the art. It is to be understood that both the foregoing general description and the following detailed description, the claims, as well as the appended drawings are exemplary and explanatory only, and are intended to provide an explanation of various embodiments of the present teachings. The specific embodiments described herein are offered by way of example only and are not meant to be limiting in any way.

### BRIEF DESCRIPTION OF THE DRAWINGS

In general, each of the FIGURES provide schematic representational illustrations of embodiments of the invention and its components. The relative location shapes, and/or sizes of objects are exaggerated and/or simplified to facilitate discussion and presentation herein.

FIG. 1 shows a schematic view of an exemplary bioreactor manifold connector body in accordance with aspects of the present invention;

FIG. 2 shows a schematic view of an exemplary bioreactor manifold system in accordance with additional aspects of the present invention; and

FIG. 3 shows a schematic view of an exemplary bioreactor manifold system in accordance with additional aspects of the present invention.

### DETAILED DESCRIPTION OF THE PREFERRED EMBODIMENTS

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about”.

Before describing the present invention in further detail, a number of terms will be defined. Use of these terms does not limit the scope of the invention but only serve to facilitate the description of the invention.

As used herein, the singular forms “a,” “an, “and “the” include plural referents unless the context clearly dictates otherwise.

As used herein the phrase, “biological samples” mean, but are not limited to, any particle(s), substance(s), extract(s), mixture, and/or assembly derived from or corresponding to one or more organisms, cells, and/or viruses. It will be apparent to one skilled in the art that cells which may be cultured in an automated cell management system comprise one or more cell types including, but not limited to, animal cells, insect cells, mammalian cells, human cells, transgenic cells, genetically engineered cells, transformed cells, cell lines, plant cells, anchorage-dependent cells, anchorage-independent cells, and other cells capable of being cultured in vitro as known in the art. The biological sample also may include additional components to facilitate analysis, such as fluid (e.g., water), buffer, culture nutrients, salt, other reagents, dyes, etc. Accordingly, the biological sample may include one or more cells disposed in a culture medium and/or another suitable fluid medium.

As used herein, the term “bioreactor” or “bioreactor vessel” refers to any apparatus, such as a large fermentation chamber, for growing organisms such as bacteria or yeast under controlled conditions for production of substances such as pharmaceuticals, antibodies, or vaccines, or for the bioconversion of organic waste.

As used herein, “cell culture” means growth, maintenance, differentiation, transfection, or propagation of cells, tissues, or their products.

As used herein the term “sensor” or “probe” means, but is not limited to, mechanical, electrical or optical sensing or probing devices that measure information such as physiologically relevant information (e.g., mixing rate, gas flow rate temperature, humidity, pressure, pH, biochemicals such as glucose, glutamine, lactic acid, ammonia, and nitrogen, biomolecules, dissolved gases such as oxygen, CO₂, and other chemical parameters, enzyme-based parameters, radiation, magnetic and other physical parameters), or other information or parameters such as spectroscopy. the sensors/probes may be optical probes which present the output in a visual manner.

As used herein the phrase “culture medium” or “culture media” means a liquid solution used to provide nutrients (e.g., vitamins, amino acids, essential nutrients, salts, and the like) and properties (e.g., similarity, buffering) to maintain living cells (or living cells in a tissue) and support their growth. Commercially available tissue culture medium is known to those skilled in the art. The phrase, “cell culture medium” as used herein means tissue culture medium that has been incubated with cultured cells in forming a cell culture; and more preferably refers to tissue culture medium that further comprises substances secreted, excreted or released by cultured cells, or other compositional and/or physical changes that occur in the medium resulting from culturing the cells in the presence of the tissue culture medium.

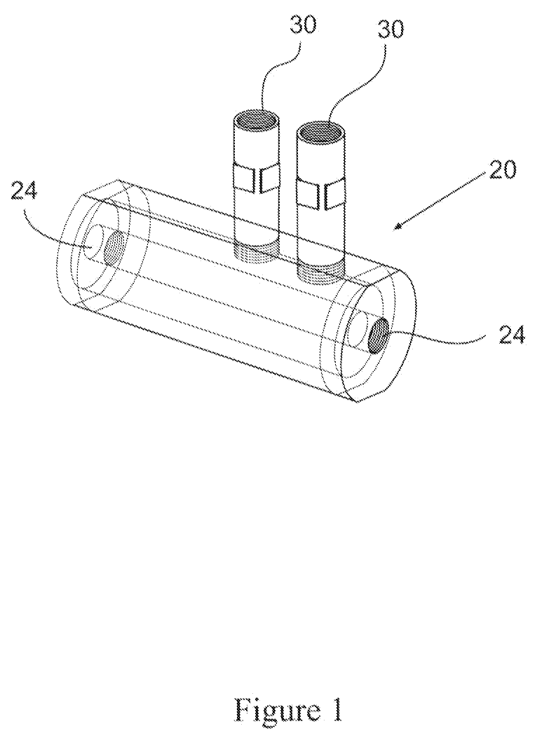

FIG. 1 shows a schematic view of an exemplary bioreactor manifold connector body (20) coupled to connectors (30), and having a sterile connector ports (24) in accordance with the present invention.

FIGS. 1-3 illustrate examples of the disposable bioreactor manifold systems (10, 70) according to certain embodiments of the invention.

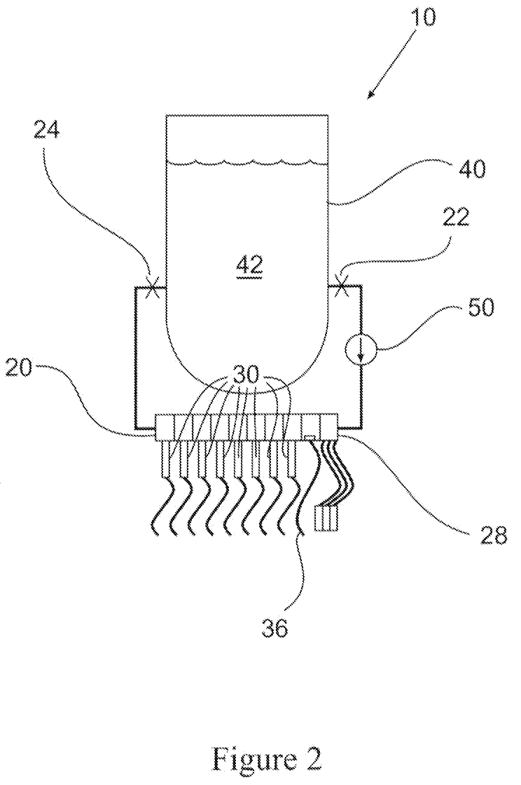

FIG. 2 shows a schematic view of a exemplary bioreactor manifold system (10) including an exemplary bioreactor manifold connector body (20) coupled to modular sensors (30), a preinstalled single use sensor (36), a recirculation pump (50), a bioreactor vessel (40) containing medium (42), sterile connectors (22, 24), and a modular medium sampler for removing and/or adding medium (28) in accordance with aspects of the present invention. It should be noted that the bioreactor manifold connector body (20) be coupled to any location on the bioreactor vessel (40), so long as that location is accessible by a bioreactor port.

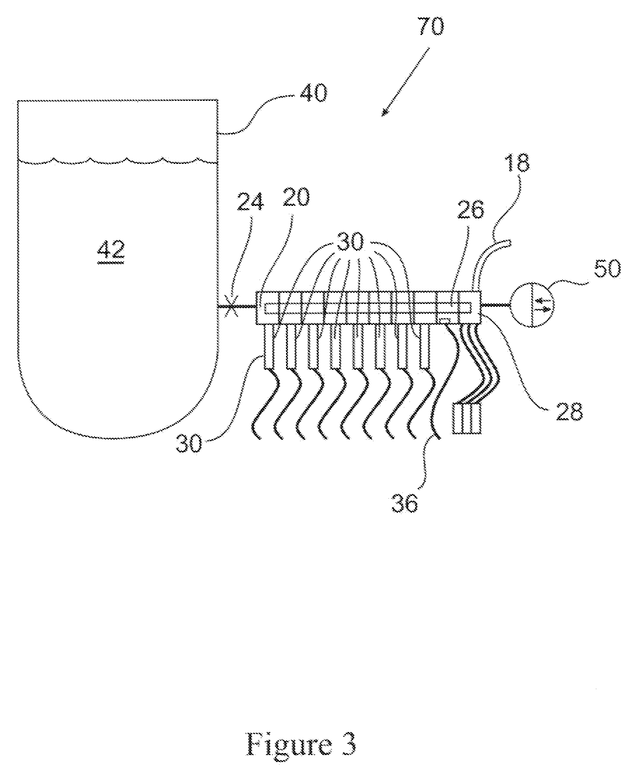

FIG. 3 shows a schematic view of a exemplary bioreactor manifold system (70) including an exemplary bioreactor manifold connector body (20) having a hollow fiber module contained therein (26), coupled to modular sensors (30), a preinstalled single use sensor (36), a recirculation pump (50), bioreactor vessel (40) containing medium (42), permeate line (18), a sterile connector (24), and an additional modular sample addition (28) in accordance with aspects of the present invention.

The bioreactor vessel (40) are preferably sterile single-use bioreactors (SUBs) manufactured from polymeric materials, such as fluoropolymers, high density polypropylene (HDPE) and specially-treated polystyrene plastic.

The disclosure set forth above may encompass multiple distinct inventions with independent utility. Although each of these inventions has been disclosed in its preferred, form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A disposable bioreactor manifold connector body comprising: a hollow housing having a generally continuous outer wall, wherein the wall defines a hollow interior chamber, an open inlet port, an open outlet port, and at least one more additional port for receiving one or more modular components.

2. The manifold of claim 1 wherein modular components are selected from the group consisting a sensor, probe, sampling component, connector, line, and combinations thereof.

3. The manifold of claim 2 wherein the modular components are disposable and sterile.

4. The manifold of claim 1 wherein the hollow housing is sterile.

5. The manifold of claim 2 wherein the sensors are mechanical, optical, electronic, or combinations thereof.

6. The manifold of claim 1 wherein the manifold consists of a material resistant to ETO.

7. The manifold of claim 1 wherein the manifold consists of a material resistant to gamma radiation.

8. The manifold of claim 2 wherein the sensor is selected from the group consisting of a pH sensor, temperature sensor, humidity sensor, pressure sensor, glucose sensor, oxygen sensor, carbon dioxide sensor, glutamine sensor, lactic acid sensor, ammonia sensor, nitrogen sensor, spectroscopy sensor, and combinations thereof.

9. A disposable bioreactor manifold system comprising:
a disposable bioreactor vessel,
a disposable manifold connector body having a hollow housing having a generally continuous outer wall, wherein the wall defines a hollow interior chamber, an open inlet port fluidly connected to the bioreactor, an open outlet port, and at least one more additional port for receiving one or more modular components,
a disposable modular component fluidly attached to the connector body,
a disposable conduit fluidly connecting the bioreactor and the manifold,
a disposable pump, and
a disposable conduit fluidly connecting the pump and bioreactor,

10. The system according to claim 9 wherein the modular components are selected from the group consisting a sensor, probe, sampling component, connector, line, and combinations thereof.

11. The system according to claim 9 further comprising a permeate line for drawing off permeate from the manifold connector body.

12. The system according to claim 9 wherein the modular components are disposable and sterile.

13. The system according to claim 9 wherein the hollow housing is sterile.

14. The system according to claim 9 wherein the sensors are mechanical, optical, electronic, or combinations thereof.

15. The system according to claim 9 wherein the manifold consists of a material resistant to ETO.

16. The system according to claim 9 wherein the manifold consists of a material resistant to gamma radiation.

17. The system according to claim 9 wherein the sensor is selected from the group consisting of a pH sensor, temperature sensor, humidity sensor, pressure sensor, glucose sensor, oxygen sensor, carbon dioxide sensor, glutamine sensor, lactic acid sensor, ammonia: sensor, nitrogen sensor, spectroscopy sensor, and combinations thereof.
